(19) United States
(12) Patent Application Publication
(10) Pub. No.: US 2026/0315441 A1
(43) Pub. Date: Oct. 15, 2026

(54) FORMULATIONS OF PENICILLIN AND METHODS FOR STABILIZING PENICILLIN

(71) Applicant: VETERINARY PHARMACY CORPORATION, St. Peter, MN (US)

(72) Inventor: Patrick John Smith, St. Peter, MN (US)

(73) Assignee: VETERINARY PHARMACY CORPORATION, St. Peter, MN (US)

(21) Appl. No.: 19/683,421

(22) Filed: May 20, 2026

Related U.S. Application Data
(63) Continuation of application No. PCT/US2024/056700, filed on Nov. 20, 2024.
(60) Provisional application No. 63/601,660, filed on Nov. 21, 2023.

(51) Int. Cl. A61K 31/43; A61K 9/08; A61K 47/12

(52) U.S. Cl. CPC A61K 31/43; A61K 9/08; A61K 47/12

(57) ABSTRACT

A formulation may include a single-phase solution, including: a penicillin, a stabilizing agent; and a water, wherein the penicillin has a potency of 80% or higher at 24 hours.

### CROSS-REFERENCE TO RELATED APPLICATIONS

This application is a continuation application of PCT International Application No. PCT/US2024/056700, filed Nov. 20, 2024, which claims priority to and the benefit of U.S. Provisional Application No. 63/601,660 filed on Nov. 21, 2023, each of which are herein incorporated by reference in their entireties.

### FIELD

The present disclosure relates generally to formulating a compound to be in a soluble form for treatment of animals.

### BACKGROUND

Penicillin is an effective antibiotic which is widely used. Penicillin is derived from Penicillium fungi, which can be used to treat and prevent various bacterial infections. Penicillin is commonly used to treat a range of infections caused by gram-positive bacteria. Penicillin can be used as a prophylactic measure to prevent infections in human patients with weakened immune systems.

### SUMMARY

The following summary is a high-level overview of various aspects and introduces some of the concepts that are further described in the Detailed Description section below. This summary is not to be used in isolation to determine the scope of the claimed subject matter. The subject matter should be understood by reference to appropriate portions of the entire specification, any or all drawings, and each claim.

The core structure of penicillin is shown below, where “R” is a variable group.

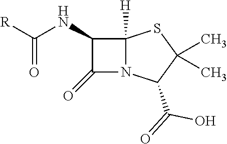

In some aspects, the techniques described herein relate to a composition including: a single-phase solution, including: a penicillin; a stabilizing agent; and a water, wherein the penicillin has a potency of 80% or higher at 24 hours.

In some aspects, the techniques described herein relate to a composition including: a single-phase solution, including: a penicillin; a pH adjusting compound; a stabilizing agent; and a water, wherein the penicillin has a potency of 80% or higher at 24 hours.

In some aspects, the single-phase solution is a clear single-phase solution.

In some aspects, the techniques described herein relate to a composition including: a colloidal solution, including: a penicillin; a stabilizing agent; and a water, wherein the penicillin has a potency of 80% or higher at 24 hours.

In some aspects, the techniques described herein relate to a composition including: a colloidal solution, including: a penicillin; a pH adjusting compound; a stabilizing agent; and a water, wherein the penicillin has a potency of 80% or higher at 24 hours.

In some aspects, the techniques described herein relate to a composition, wherein the stabilizing agent includes a potassium sorbate.

In some aspects, the techniques described herein relate to a composition, wherein the stabilizing agent includes EDTA, sorbic acid, butyric acid salts, propionic acid, maleic acid, potassium carbonate, sodium carbonate, arginine salts, urea, or a combination thereof.

In some aspects, the techniques described herein relate to a composition, wherein the penicillin includes penicillin G.

In some aspects, the techniques described herein relate to a composition, wherein the penicillin is penicillin G.

In some aspects, the techniques described herein relate to a composition, wherein the pH of the single-phase solution is outside a range of pH 5-8.

In some aspects, the techniques described herein relate to a composition, wherein the pH of the single-phase solution is in a range of pH 9 to pH 9.7.

In some aspects, the techniques described herein relate to a composition, wherein the pH of the colloidal solution is outside a range of pH 5-8.

In some aspects, the techniques described herein relate to a composition, wherein the pH of the colloidal solution is in a range of pH 9 to pH 9.7.

In some aspects, the techniques described herein relate to a method for preparing a single-phase solution, the method including: obtaining a mixture, wherein the mixture includes: a penicillin; and a stabilizing agent, obtaining a water; combining the mixture and the water to dissolve the mixture in the water, wherein after 24 hours at room temperature, a potency of the penicillin is at least 80%.

In some aspects, the techniques described herein relate to a method for preparing a colloidal solution, the method including: obtaining a mixture, wherein the mixture includes: a penicillin; and a stabilizing agent, obtaining a water; combining the mixture and the water to dissolve the mixture in the water, wherein after 24 hours at room temperature, a potency of the penicillin is at least 80%.

In some aspects, the techniques described herein relate to a method, wherein after 24 hours at room temperature, the potency of the penicillin is at least 90%.

In some aspects, the techniques described herein relate to a method, wherein after 24 hours at room temperature, the potency of the penicillin is from 90% to 97%.

In some aspects, the techniques described herein relate to a method wherein the pH of the single-phase solution is outside a range of pH 5-8.

In some aspects, the techniques described herein relate to a method, wherein the pH of the single-phase solution is in a range of—from pH 9 to pH 9.7.

In some aspects, the techniques described herein relate to a method wherein the pH of the colloidal solution is outside a range of pH 5-8.

In some aspects, the techniques described herein relate to a method, wherein the pH of the colloidal solution is in a range of from pH 9 to pH 9.7.

In some aspects, the techniques described herein relate to a method, wherein the stabilizing agent includes a potassium sorbate.

In some aspects, the techniques described herein relate to a method, wherein the stabilizing agent includes EDTA, sorbic acid, butyric acid salts, propionic acid, maleic acid, potassium carbonate, sodium carbonate, arginine salts, urea, or a combination thereof.

In some aspects, the techniques described herein relate to a method, wherein the penicillin includes penicillin G.

In some aspects, the techniques described herein relate to a method, wherein the penicillin is penicillin G.

### DETAILED DESCRIPTION

Penicillin as used herein includes penicillin G, penicillin V, penicillin, and/or a combination thereof. The embodiments disclosed herein may have application to animals, mammals, etc. The embodiments disclosed herein are directed toward treatment(s) of animals, mammals, and even humans. The various treatments can include, according to some embodiments, bacterial infections.

Penicillin G (a.k.a. benzylpenicillin) has a structure of penicillin, wherein the “R” is a benzyl group, as shown below.

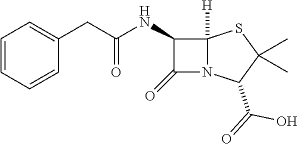 [SMILES: CC1(C)S[C@@H]2[C@H](NC(=O)Cc3ccccc3)C(=O)N2[C@H]1C(=O)O]

Penicillin G belongs to the β-lactam class of antibiotics, wherein these molecules are able to covalently bond to proteins that are involved in bacteria's cellular wall synthesis, resulting in a disruption of the cell wall construction. Penicillin G includes a thiazolidine β-lactam moiety named 6-aminopenicillanic acid.

The embodiments and examples discussed herein are directed toward manufacturing of a concentrated medicated stock solution containing penicillin. This stock solution can then be metered out at a specific dose for all day delivery of the medication (i.e., penicillin) for either a single animal or entire herd of animals. For example, the embodiments and examples herein are directed towards making concentrated stock solution(s) of penicillin, which is then metered out at, a particular dilution factor and/or within a range of specified dilution factors. The embodiments of the stock solution can be made either from a pre concentrated blend of penicillin and stabilizers or where penicillin is added first to the solution followed shortly by the stabilizer component. The blend of some components can either be in powder or liquid form. According to some embodiments, the medicated stock solution is made by mixing with water which can be found in a field near the animal or herd of animals. It is generally understood that “water” used in solubility tests are purified water, distilled water, sterile water, or high-quality water with extremely low amounts of contaminants (e.g., what is generally known as “hard water” is not or cannot be used). For example, “water” used for mixing with penicillin in injections require sterile water, and one of skill in the art understands that “water” used in injections cannot be field sourced water. In contrast, some of the embodiments in this disclosure have been tested with various water sourced across the United States of America. It has been determined that, according to some embodiments, the “water” can be water sources from the field, a well, or any other convenient sources, where the “water” is not purified (e.g., distilled) water. That is, according to some embodiments, the term “water” means any water. According to some embodiments, the term “water” does not include sterile water. According to some embodiments, the term “water” does not include purified water (e.g., distilled water). As such, treatment of animals (e.g., swine, etc.) in the field can be improved by the embodiments herein, as the high potency can be achieved and maintained for longer periods of time, even when convenient water sources are used.

According to some embodiments, a stabilizing agent is a compound which stabilizes penicillin in water such that the penicillin maintains high potency over time (e.g., at 24 hours or longer, stored at room temperature). That is, the stabilizing agent, according to some embodiments, has a specific function in the embodied methods and formulations of stabilizing the potency of the penicillin mixed in water.

According to some embodiments, a hydrotrope can be added to a formulation. A hydrotrope is a compound that increases the solubility of hydrophobic compounds in water. The hydrotrope includes a hydrophilic part and a hydrophobic part, but unlike surfactants, the hydrotrope does not form micelles. Instead, hydrotrope act by disrupting the structure of water, reducing its surface tension and making it easier for hydrophobic substances to dissolve. According to some embodiments, formulations and/or methods disclosed herein include a hydrotrope compound. According to some embodiments, the stabilizing agent includes a hydrotrope compound. An example of a hydrotrope compound is Niacinamide.

According to some embodiments, the formulations and methods disclosed herein do not include sterile water.

According to some embodiments, penicillin is used, which is an antibiotic that has impressive coverage over many organisms that affect swine and other animals in the veterinary industry. According to some embodiments, penicillin G is used, which is an antibiotic that has impressive coverage over many organisms that affect swine and other animals in the veterinary industry.

There are at least two major challenges with penicillin: (1) Chemical stability over time; and (2) Inherently insoluble nature. These three challenges are discussed in detail below.

### Chemical Stability

Penicillin's stability in an aqueous solution can be affected by concentration of penicillin, temperature, pH, time, and any combination(s) thereof. For example, penicillin begins to break down due to hydrolysis of the molecule. This breakdown is enhanced by factors such as pH, concentration, and/or temperature.

For example, penicillin G is sensitive to temperature. Further, penicillin G is known to have the highest stability between a pH of 5-8. It has been reported that penicillin G for injection diluted in sodium chloride injection or dextrose injection can be made chemically stable for a period when (1) stored at the low temperature of 5° C.; and (2) pH of 5.28 to 6.73 (required). Penicillin G is known to have increased degradation as temperature is increased, for example, from 1.92%/hour (at 5° C.) to 3.29%/hour (at 37° C.). Further, penicillin is known to be very unstable outside of the pH range of 5-8.

### EMBODIMENTS AND PROPERTIES

According to some embodiments, formulations of penicillin in solution include potassium sorbate (or other salts or chemically similar structures). According to some embodiments, formulations of penicillin in solution include potassium sorbate (or other salts or chemically similar structures). Potassium sorbate can be effective against gram-negative bacteria (for example, Escherichia coli). Combining it with penicillin could add synergism to gain back activity against gram-negative bacteria.

Surprisingly and unexpectedly, formulations that include penicillin and potassium sorbate, when added in varying ratios, were able to solubilize the penicillin at higher-than-normal concentrations. Embodiments of the formulations also had surprisingly unexpected properties. According to the embodiments, the pH achieved at solubilization was at what was generally understood to be an insoluble range for normal penicillin solutions. For example, according to the embodiments, clear solutions were achieved at pH of 7.5 to 7.6. Because of the lower pH of these clear solutions, it was theorized that the penicillin, according to the embodiments, could lead to better stability of the product compared to other known methods of solubilization. Potency studies demonstrated that the formulations according to some embodiments had at least a 7% higher potency at 24 hours, and up to a 13-15% potency difference at 48 hours. Some embodiments of the penicillin solutions maintained greater than 80% potency at 24 hours. According to some embodiments, the term “high potency” can be a potency at or above 80% at 24 hours at room temperature. According to some embodiments, the term “high potency” can be a potency at or above 84% at 24 hours at room temperature. The term “high potency” can be a potency at or above 85% at 24 hours at room temperature, according to some embodiments. The term “high potency” can be a potency at or above 87% at 24 hours at room temperature, according to some embodiments. The term “high potency” can be a potency at or above 89% at 24 hours at room temperature, according to some embodiments. The term “high potency” can be a potency at or above 90% at 24 hours at room temperature, according to some embodiments. The term “high potency” can be a potency above 90% at 24 hours at room temperature, according to some embodiments. The term “high potency” can be a potency at or above 91% at 24 hours at room temperature, according to some embodiments. The term “high potency” can be a potency above 91% at 24 hours at room temperature, according to some embodiments. The term “high potency” can be a potency at or above 92% at 24 hours at room temperature, according to some embodiments. The term “high potency” can be a potency above 92% at 24 hours at room temperature, according to some embodiments. The term “high potency” can be a potency at or above 93% at 24 hours at room temperature, according to some embodiments. The term “high potency” can be a potency above 93% at 24 hours at room temperature, according to some embodiments. The term “high potency” can be a potency at or above 94% at 24 hours at room temperature, according to some embodiments. The term “high potency” can be a potency above 94% at 24 hours at room temperature, according to some embodiments. The term “high potency” can be a potency at or above 95% at 24 hours at room temperature, according to some embodiments. The term “high potency” can be a potency above 95% at 24 hours at room temperature, according to some embodiments. The term “high potency” can be a potency at or above 96% at 24 hours at room temperature, according to some embodiments. The term “high potency” can be a potency above 96% at 24 hours at room temperature, according to some embodiments. The term “high potency” can be a potency at or above 97% at 24 hours at room temperature, according to some embodiments. The term “high potency” can be a potency maximum of 97% at 24 hours at room temperature, according to some embodiments. The term “high potency” can be a potency of 80% to 97% at 24 hours at room temperature, according to some embodiments. The term “high potency” can be a potency of 90% to 97% at 24 hours at room temperature, according to some embodiments.

According to some embodiments, it was also found that potassium sorbate surprisingly and unexpectedly lowers the requirement of alkaline base or other alkaline solubilizers by a minimum of 10× than what was generally understood to be required.

### Examples of Formulations

The following are formulation examples according to the various embodiments disclosed herein. It is to be understood that the examples provided herein are not necessarily limiting. In all of the Formulations below, the powder blend(s) immediately form a clear single-phase colloidal solution. In all of the Formulations below, penicillin powder was at 99.6% potency. It should be noted that the penicillin percentage is given as the penicillin base. The penicillin trihydrate form is what has been used to compound these various Formulations.

Comparative Example Formulation (C-1):

| | |
|---|---|
| Penicillin G Potassium | 500,000,000 units |
| Water | Q.S. to 9462.50 ml |

In this comparative example, tests revealed that the potency of penicillin G potassium averaged around 80-85% potency at 24 hours. As with all known beta lactams, there was significant degradation that occurred in the water solution over time. As such, a fresh stock solution should be prepared every 12 hours with this product to maintain >90% potency.

Formulation 1:

| | |
|---|---|
| Penicillin G Potassium | 500,000,000 units |
| Potassium Sorbate | 800 g (8.45% wt/vol) |
| Water | Q.S. to 9462.50 ml |

In this Formulation, utilizing potassium sorbate as a stabilizer surprisingly and unexpectedly resulted in a degradation rate of ~3.5% at 24 hours. As a comparison, the degradation rate is 10-15% for the Comparative Example Formulation C-1. Thus, Formulation 1 represents a significant and unexpected breakthrough.

Generally, it had been understood that the minimum degradation pH point of Penicillin is pH 6.5. This is because very strong alkaline solutions can cause hydrolyzation of penicillin transforming it to penicilloic acid. However, the following Formulations show surprising and unexpected stability outside of the pH ranges for penicillin stability.

Comparative Example Formulation (C-2):

| | |
|---|---|
| Penicillin G Potassium | 5,602,772 million units |
| Water | Q.S. to 100 ml |

This was a control sample to test the pure penicillin G potassium in stock solution environment. Potency testing at 23 hours showed 89.05% potency. Other potency tests showed potency in a range of 80-84% at the 24-hour period.

Formulation 2:

| | |
|---|---|
| Penicillin G Potassium | 500,000,000 units |
| Potassium Sorbate | 806 g (8.19% wt/vol) |
| Water | Q.S. to 9841 ml |
| pH: | 9.0 to 9.7 |

The Formulation showed 97.1% potency at 24 hours.

Formulation 3:

| | |
|---|---|
| Penicillin G Potassium | 500,000,000 units |
| Potassium Sorbate | 206.5 g (2.10% wt/vol) |
| Water | Q.S. to 9841 ml |
| pH: | 9.0 to 9.7 |

The Formulation showed 96.5% potency at 24 hours.

Formulation 4:

| | |
|---|---|
| Penicillin G Potassium | 5,377,730 million units |
| Potassium Sorbate | 2.1160 g (2.12% wt/vol) |
| Water | Q.S. to 100 ml |

This Formulation showed 99.79% potency at 24 hours. This result was surprising and unexpected. This Formulation demonstrated a significant improvement to the

Comparative Example Formulation (C-2).
Comparative Example Formulation (C-3):

| | |
|---|---|
| Penicillin G Potassium | 132,100 units / 1 mL |
| Water | Q.S. to 100 ml |

This control formulation set an understanding of the stability for pure penicillin G potassium in stock solution environment. Potency testing at 24 hours showed a 79.2% potency.

Formulation 5:

| | |
|---|---|
| Penicillin G Potassium | 132100 units / 1 mL |
| Potassium Sorbate | 2.1160 g (2.12% wt/vol) |
| Water | Q.S. to 100 ml |

This Formulation tested at 99.0% potency at 24 hours. This surprising and unexpected result demonstrated a significant improvement to the Comparative

Example Formulation C-3.
Comparative Example Formulation (C-4).

| | |
|---|---|
| Penicillin G Potassium | 53,108.44 units/ 1 mL |
| Water | Q.S. to 1000 ml |

After 168 hours, there was no detectable peak or penicillin G product. Thus, after 168 hours, it can be understood that there was complete hydrolysis of the penicillin molecule.

Formulation 6:

| | |
|---|---|
| Penicillin G Potassium | 53,108.44 units / 1 mL |
| Potassium Sorbate | 22.0 g (2.20% wt/vol) |
| Water | Q.S. to 1000 ml |

At 168 hours, Formulation 6 showed potency of 88.5%. As compared to Comparative Example Formulation C-4, this enhanced stability was surprising and unexpected.

Further tests were conducted over a 192-hour time period. Two different products were tested.

Formulation 7:

| | | |
|---|---|---|
| Penicillin G Potassium | 53,108.44 | units/1 mL |
| Potassium Sorbate | 22.12 | g |

| | |
|---|---|
| Water | Q.S. to 1000 ml |

At the 192-hour mark, potency was at 91.3%.

Formulation 8:

| | | |
|---|---|---|
| Penicillin G Potassium | 53,108.44 | units /I mL |
| Potassium Sorbate | 210 | g |

| | |
|---|---|
| Water | Q.S. to 12,112 ml |

At the 192-hour mark, potency was at 96.7%. This Formulation showed an extremely stable solution at a prolonged period of time.

Another molecule that can be added to any one or more formulations envisioned herein is EDTA. It is believed that EDTA can enhance activity against gram-negative bacteria. The EDTA is a chelating molecule which is expected to enhance metal cation binding that can prevent common mechanisms of resistance in gram-negative efflux pumps. It is believed that by adding the EDTA with or without potassium sorbate in the formulation can affect (e.g., enhance) the spectrum of activity for penicillin and other beta lactams. Other stabilizers can be or include sorbic acid, butyric acid salts, propionic acid, maleic acid, potassium carbonate, sodium carbonate, arginine salts, urea, or a combination thereof.

The Formulations discussed above exemplify how utilizing potassium sorbate was able to create extremely stable compounds of penicillin in aqueous solutions. An advantageous application of this, along with increased spectrum of antibacterial activity, is the ability to mix with other agents to lower the amount of potassium sorbate in the formulation but achieving a greater than 90% potency of penicillin at 24 hours (or longer). While some experiments can be conducted using pure powder forms of penicillin, formulations can use penicillin powder derived from capsule materials in a pharmacy compounding process. According to embodiments of the compounding process, the process includes obtaining penicillin capsules; obtaining ground materials by grounding the penicillin capsules; sifting the ground materials to remove as much excess capsule material as possible; and then obtaining powdered penicillin from the sifting. According to some embodiments, penicillin can be obtained already in a powder form, such as a powder blend form. The powder is then tested via HPLC assay to assess potency of penicillin base to help with adding correct amount of powder to desired stock solution concentration. There was a concern with this process that some capsule fragments would still remain and not be dissolved in water. Generally, it was expected that these capsule fragments would settle to the bottom of the stock solution, and could be problematic for water medicator systems. Surprisingly and unexpectedly, it was found that utilizing potassium sorbate with the compounded product resulted in a dissolution of the capsule fragments without altering the stability of the product.

### Aspects:

Aspect 1. A composition comprising: a clear single-phase solution, including: a penicillin; a stabilizing agent; and a water, wherein the penicillin has a potency of 80% or higher at 24 hours.

Aspect 2. The composition of Aspect 1, wherein the stabilizing agent comprises a potassium sorbate.

Aspect 3. The composition according to any of Aspects 1-2, wherein the stabilizing agent comprises EDTA, sorbic acid, butyric acid salts, propionic acid, maleic acid, potassium carbonate, sodium carbonate, arginine salts, urea, or a combination thereof.

Aspect 4. The composition according to any of Aspects 1-3, wherein the penicillin includes penicillin G.

Aspect 5. The composition according to any of Aspects 1-3, wherein the penicillin is penicillin G.

Aspect 6. The composition according to any of Aspects 1-5, wherein the pH of the single-phase solution is outside a range of pH 5-8.

Aspect 7. The composition according to any of Aspects 1-5, wherein the pH of the single-phase solution is in a range of pH 9 to pH 9.7.

Aspect 8. A method for preparing a single-phase solution, the method comprising: obtaining a mixture, wherein the mixture comprises: a penicillin; and a stabilizing agent, obtaining a water; combining the mixture and the water to dissolve the mixture in the water, wherein after 24 hours at room temperature, a potency of the penicillin is at least 80%.

Aspect 9. The method of Aspect 8, wherein after 24 hours at room temperature, the potency of the penicillin is at least 90%.

Aspect 10. The method of Aspect 8, wherein after 24 hours at room temperature, the potency of the penicillin is from 90% to 97%.

Aspect 11. The method according to any of Aspects 8-10, wherein the pH of the single-phase solution is outside a range of pH 5-8.

Aspect 12. The method to any of Aspects 8-10, wherein the pH of the single-phase solution is in a range of from pH 9 to pH 9.7.

Aspect 13. The method to any of Aspects 8-12, wherein the stabilizing agent comprises a potassium sorbate.

Aspect 14. The method to any of Aspects 8-13, wherein the stabilizing agent comprises EDTA, sorbic acid, butyric acid salts, propionic acid, maleic acid, potassium carbonate, sodium carbonate, arginine salts, urea, or a combination thereof.

Aspect 15. The method to any of Aspects 8-14, wherein the penicillin includes penicillin G.

Aspect 16. The method to any of Aspects 8-14, wherein the penicillin is penicillin G.

Among those benefits and improvements that have been disclosed, other objects and advantages of this disclosure will become apparent from the following description taken in conjunction with the accompanying figures. Detailed embodiments of the present disclosure are disclosed herein; however, it is to be understood that the disclosed embodiments are merely illustrative of the disclosure that may be embodied in various forms. In addition, each of the examples given regarding the various embodiments of the disclosure which are intended to be illustrative, and not restrictive.

The terminology used herein is intended to describe embodiments and is not intended to be limiting. The terms “a,” “an,” and “the” include the plural forms as well, unless clearly indicated otherwise. The terms “comprises” and/or “comprising,” when used in this Specification, specify the presence of the stated features, integers, steps, operations, elements, and/or components, but do not preclude the presence or addition of one or more other features, integers, steps, operations, elements, and/or components. As used herein, the term “based on” is not exclusive and allows for being based on additional factors not described, unless the context clearly dictates otherwise. In addition, the meaning of “in” includes “in” and “on.”

It is to be understood that changes may be made in detail, especially in matters of the construction materials employed and the shape, size, and arrangement of parts without departing from the scope of the present disclosure. This Specification and the embodiments described are examples, with the true scope and spirit of the disclosure being indicated by the claims that follow.

What is claimed is:

1. A composition comprising:
a single-phase solution, including:
a penicillin;
a stabilizing agent; and
a water,
wherein the penicillin has a potency of 80% or higher at 24 hours.

2. The composition of claim 1, wherein the stabilizing agent comprises a potassium sorbate.

3. The composition of claim 1, wherein the stabilizing agent comprises EDTA, sorbic acid, butyric acid salts, propionic acid, maleic acid, potassium carbonate, sodium carbonate, arginine salts, urea, or a combination thereof.

4. The composition of claim 1, wherein the penicillin includes penicillin G.

5. The composition of claim 1, wherein the penicillin is penicillin G.

6. The composition of claim 1, wherein the pH of the single-phase solution is outside a range of pH 5-8.

7. The composition of claim 1, wherein the pH of the single-phase solution is in a range of pH 9 to pH 9.7.

8. A method for preparing a single-phase solution, the method comprising:
obtaining a mixture,
wherein the mixture comprises:
a penicillin; and
a stabilizing agent,
obtaining a water;
combining the mixture and the water to dissolve the mixture in the water,
wherein after 24 hours at room temperature, a potency of the penicillin is at least 80%.

9. The method of claim 8, wherein after 24 hours at room temperature, the potency of the penicillin is at least 90%.

10. The method of claim 8, wherein after 24 hours at room temperature, the potency of the penicillin is from 90% to 97%.

11. The method of claim 8 wherein the pH of the single-phase solution is outside a range of pH 5-8.

12. The method of claim 8, wherein the pH of the single-phase solution is in a range of from pH9 to pH 9.7.

13. The method of claim 8, wherein the stabilizing agent comprises a potassium sorbate.

14. The method of claim 8, wherein the stabilizing agent comprises EDTA, sorbic acid, butyric acid salts, propionic acid, maleic acid, potassium carbonate, sodium carbonate, arginine salts, urea, or a combination thereof.

15. The method of claim 8, wherein the penicillin includes penicillin G.

16. The method of claim 8, wherein the penicillin is penicillin G.
